(19) United States
(12) Patent Application Publication
(10) Pub. No.: US 2007/0117978 A1
(43) Pub. Date: May 24, 2007

(54) Cardiotonic compounds with inhibitory activity against beta-adrenergic receptors and phosphodiesterase

(75) Inventors: Gregory Hamilton, Catonsville, MD (US); Harry Leighton, Boston, MA (US)

Correspondence Address: SEED INTELLECTUAL PROPERTY LAW GROUP PLLC, 701 FIFTH AVE, SUITE 5400, SEATTLE, WA, 98104, US

(73) Assignee: ARTESIAN THERAPECUTICS, INC, GAITHERSBURG MARYLAND (US)

(21) Appl. No.: 10/547,930

(22) PCT Filed: Dec. 23, 2003
(86) PCT No.: PCT/US03/41031

§ 371 Date: Dec. 13, 2006

(51) Int. Cl. A61K 31/519; A61K 31/502; A61K 31/501; A61K 31/4745; A61K 31/4704; C07D 487/04; C07D 403/02

(52) U.S. Cl. 544/235; 544/238; 544/256; 544/281; 544/250; 546/157; 546/82; 548/324.1; 548/544

(57) ABSTRACT

The present invention provides compounds possessing inhibitory activity against (3-adrenergic receptors and phosphodiesterase (PDE), including type 3 phosphodiesterase (PDE-3). The present invention further provides pharmaceutical compositions comprising such compounds, methods of preparing such compounds, and methods of using such compounds for regulating calcium homeostasis, for treating a disease, disorder or condition in which disregulation of calcium homeostasis is implicated and for treating cardiovascular disease, stroke, epilepsy, an ophthalmic disorder or migraine.

This application claims the benefit of U.S. Provisional Patent Application No. 60/435,524, filed Dec. 23, 2002, the entire contents of which are herein incorporated by reference.

Congestive heart failure affects an estimated 4.8 million Americans with over 400,000 new cases diagnosed each year. Despite incremental advances in drug therapy, the prognosis for patients with advanced heart failure remains poor with annual mortality exceeding 40 percent. Although heart transplantation is an effective therapy for patients with advanced heart failure, less than 2,200 heart transplants are performed annually due to a limited supply of donor organs. Recent analyses indicate that further increases in the incidence and prevalence of advanced heart failure are likely, highlighting the pressing need for novel and effective therapeutic strategies.

During heart failure, there is an alteration of calcium homeostasis, including impaired sarcoplasmic reticulum calcium re-uptake, increased basal (diastolic) calcium levels, decreased peak (systolic) calcium and reduced rate of calcium transients, resulting in a decreased force of contraction and a slowing of relaxation. The end results of these abnormalities in calcium homeostasis are depressed contractile function (decreased contractility and cardiac output), impaired ventricular relaxation, and myocyte loss via ischemia and/or apoptosis-related mechanisms. Disregulation of calcium homeostasis has also been implicated in a number of other disease states, including stroke, epilepsy, ophthalmic disorders, and migraine.

Beta-adrenergic blocking agents are common therapy for patients with mild to moderate chronic heart failure (CHF). Some patients on β-blockers may subsequently decompensate, however, and would need acute treatment with a positive inotropic agent. Phosphodiesterase inhibitors (PDEI), such as milrinone or enoximone, retain their full hemodynamic effects in the face of beta-blockade, because the site of PDEI action (cAMP) is downstream of the β-adrenergic receptor, and because β-antagonism reverses receptor pathway desensitization changes, which are detrimental to PDEI response.

### SUMMARY OF THE INVENTION

The present invention provides compounds possessing inhibitory activity against β-adrenergic receptors and phosphodiesterase (PDE), including type 3 phosphodiesterase (PDE-3). The present invention further provides pharmaceutical compositions comprising such compounds, methods of preparing such compounds, and methods of using such compounds for regulating calcium homeostasis, for treating a disease, disorder or condition in which disregulation of calcium homeostasis is implicated, and for treating cardiovascular disease, stroke, epilepsy, an ophthalmic disorder or migraine.

### DETAILED DESCRIPTION

### Definitions

“Alkyl” refers to a saturated straight or branched chain hydrocarbon radical. Examples include without limitation methyl, ethyl, propyl, iso-propyl, butyl, iso-butyl, tert-butyl, n-pentyl and n-hexyl.

“Alkenyl” refers to an unsaturated straight or branched chain hydrocarbon radical comprising at least one carbon to carbon double bond. Examples include without limitation ethenyl, propenyl, iso-propenyl, butenyl, iso-butenyl, tert-butenyl, n-pentenyl and n-hexenyl.

“Alkynyl” refers to an unsaturated straight or branched chain hydrocarbon radical comprising at least one carbon to carbon triple bond. Examples include without limitation ethynyl, propynyl, iso-propynyl, butynyl, iso-butynyl, tert-butynyl, pentynyl and hexynyl.

“Cycloalkyl” refers to a cyclic alkyl radical. Examples include without limitation cyclobutyl, cycopentyl, cyclohexyl, cycloheptyl and cyclooctyl.

“Cycloalkenyl” refers to a cyclic alkenyl radical. Examples include without limitation cyclopentenyl, cyclohexenyl, cycloheptenyl and cyclooctenyl.

“Alkoxy” refers to an alkyl group bonded through an oxygen linkage.

“Alkenoxy” refers to an alkenyl group bonded through an oxygen linkage.

“Alkylthio” refers to a sulfur substituted alkyl radical.

“Aryl” refers to a cyclic aromatic hydrocarbon moiety having one or more closed ring(s). Examples include without limitation phenyl, benzyl, naphthyl, anthracenyl, phenanthracenyl and biphenyl.

“Heteroaryl” refers to a cyclic aromatic moiety having one or more closed rings with one or more heteroatom(s) (for example, sulfur, nitrogen or oxygen) in at least one ring. Examples include without limitation pyrryl, furanyl, thienyl, pyridinyl, oxazolyl, thiazolyl, benzofuranyl, benzothienyl, benzofuranyl and benzothienyl.

“Halo” refers to a fluoro, chloro, bromo or iodo radical.

“Isosteres” refer to elements, functional groups, substituents, molecules or ions having different molecular formulae but exhibiting similar or identical physical properties. For example, tetrazole is an isostere of carboxylic acid because it mimics the properties of carboxylic acid even though they have different molecular formulae. Typically, two isosteric molecules have similar or identical volumes and shapes. Ideally, isosteric molecules should be isomorphic and able to co-crystallize. Other physical properties that isosteric molecules usually share include boiling point, density, viscosity and thermal conductivity. However, certain properties may be different: dipolar moments, polarity, polarization, size and shape since the external orbitals may be hybridized differently. The term “isosteres” encompasses “bioisosteres.”

“Bioisosteres” are isosteres that, in addition to their physical similarities, share some common biological properties. Typically, bioisosteres interact with the same recognition site or produce broadly similar biological effects.

“Substituted phenyl” refers to a phenyl that is substituted with one or more substituent(s). Examples of such substituent(s) include without limitation C₁-C₆ alkyl, C₂-C₆ alkenyl, C₂-C₆ alkynyl, C₁-C₆ alkoxy, C₂-C₆ alkenyloxy, phenoxy, benzyloxy, hydroxy, carboxy, hydroperoxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, disilanyl, siloxy, silyl, silylene and carbocyclic and heterocyclic moieties.

“Effective amount” refers to the amount required to produce a desired effect, for example, regulating calcium homeostasis, treating a disease, condition in which disregulation of calcium homeostasis is implicated, treating cardiovascular disease, stroke, epilepsy, an ophthalmic disorder or migraine, or inhibiting a β-adrenergic receptor and/or PDE, including PDE-3.

“Metabolite” refers to a substance produced by metabolism or by a metabolic process.

“Pharmaceutically acceptable carrier” refers to a pharmaceutically acceptable material, composition or vehicle, such as a liquid or solid filler, diluent, excipient or solvent encapsulating material, involved in carrying or transporting the subject compound from one organ, or portion of the body, to another organ or portion of the body. Each carrier is “acceptable” in the sense of being compatible with the other ingredients of the formulation and suitable for use with the patient. Examples of materials that can serve as a pharmaceutically acceptable carrier include without limitation: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically acceptable equivalent” includes, without limitation, pharmaceutically acceptable salts, hydrates, solvates, metabolites, prodrugs and isosteres. Many pharmaceutically acceptable equivalents are expected to have the same or similar in vitro or in vivo activity as the compounds of the invention.

“Pharmaceutically acceptable salt” refers to an acid or base salt of the inventive compounds, which salt possesses the desired pharmacological activity and is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

“Prodrug” refers to a derivative of the inventive compounds that undergoes biotransformation, such as metabolism, before exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds using conventional methods, such as that described in BURGER'S MEDICINAL CHEMISTRY AND DRUG CHEMISTRY, Fifth Ed., Vol. 1, pp. 172-178, 949-982 (1995).

“Isomers” refer to compounds having the same number and kind of atoms, and hence the same molecular weight, but differing with respect to the arrangement or configuration of the atoms.

“Stereoisomers” refer to isomers that differ only in the arrangement of the atoms in space.

“Diastereoisomers” refer to stereoisomers that are not mirror images of each other.

Diastereoisomers occur in compounds having two or more asymmetric carbon atoms; thus, such compounds have 2ⁿ optical isomers, where n is the number of asymmetric carbon atoms.

“Enantiomers” refers to stereoisomers that are non-superimposable mirror images of one another.

“Enantiomer-enriched” refers to a mixture in which one enantiomer predominates.

“Racemic” refers to a mixture containing equal parts of individual enantiomers.

“Non-racemic” refers to a mixture containing unequal parts of individual enantiomers.

“Animal” refers to a living organism having sensation and the power of voluntary movement, and which requires for its existence oxygen and organic food. Examples include, without limitation, members of the human, equine, porcine, bovine, murine, canine and feline species. In the case of a human, an “animal” may also be referred to as a “patient.”

“Mammal” refers to a warm-blooded vertebrate animal.

“Calcium homeostasis” refers to the internal equilibrium of calcium in a cell.

“Cardiovascular disease” refers to a disease of the heart, blood vessels or circulation.

“Heart failure” refers to the pathophysiologic state in which an abnormality of cardiac function is responsible for the failure of the heart to pump blood at a rate commensurate with the requirements of the metabolizing tissues.

“Congestive heart failure” refers to heart failure that results in the development of congestion and edema in the metabolizing tissues.

“Hypertension” refers to elevation of systemic blood pressure.

“SA/AV node disturbance” refers to an abnormal or irregular conduction and/or rhythm associated with the sinoatrial (SA) node and/or the atrioventricular (AV) node.

“Arrhythmia” refers to abnormal heart rhythm. In arrhythmia, the heartbeats may be too slow, too fast, too irregular or too early. Examples of arrhythmia include, without limitation, bradycardia, fibrillation (atrial or ventricular) and premature contraction.

“Hypertrophic subaortic stenosis” refers to enlargement of the heart muscle due to pressure overload in the left ventricle resulting from partial blockage of the aorta.

“Angina” refers to chest pain associated with partial or complete occlusion of one or more coronary arteries in the heart.

“Treating” refers to: (i) preventing a disease, disorder or condition from occurring in an animal that may be predisposed to the disease, disorder and/or condition but has not yet been diagnosed as having it; (ii) inhibiting a disease, disorder or condition, i.e., arresting its development; and/or (iii) relieving a disease, disorder or condition, i.e., causing regression of the disease, disorder and/or condition.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

### Compounds

The present invention provides a compound of formula I: 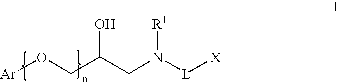
or a pharmaceutically acceptable equivalent, an isomer or a mixture of isomers thereof, wherein:

n is 0 or 1;

Ar is an aryl or heteroaryl radical, which aryl or heteroaryl radical is optionally substituted with 1 to 3 substituent(s) selected from R², R³ and R⁴;

R¹ is hydrogen, C₁-C₈ alkyl, C₂-C₈ alkenyl, C₂-C₈ alkynyl, C₃-C₈ cycloalkyl or C₃-C₈ cycloalkenyl;

R², R³ and R⁴ are independently cyano, nitro, halogen, hydrogen, trifluoromethyl, acylaminoalkyl, NR⁵, —NHSO₂R¹, —NHCONHR¹, C₁-C₄ alkoxy, C₁-C₄ alkylthio, C₁-C₈ alkyl, C₂-C₈ alkenyl or C₂-C₈ alkynyl, wherein one or more carbon(s) of said alkyl, alkenyl or alkynyl chain is/are optionally replaced with O, S, SO₂, or NR⁵, and/or optionally substituted with carbonyl oxygen or hydroxyl;

L is C₁-C₁₂ alkyl, C₂-C₁₂ alkenyl or C₂-C₁₂ alkynyl, wherein one or more carbon(s) of said alkyl, alkenyl or alkynyl is/are optionally replaced with O, S, SO₂, or NR⁵, and/or optionally substituted with carbonyl oxygen or hydroxyl;

R⁵ is hydrogen, a lone pair of electrons, C₁-C₈ alkyl, C₂-C₈ alkenyl or C₃-C₈ alkynyl, which alkyl, alkenyl or alkynyl is optionally substituted with phenyl or substituted phenyl;

X is a moiety of formula A, B, C, D, E, F, G, H, I, J, K, L, M, N, O, P or Q 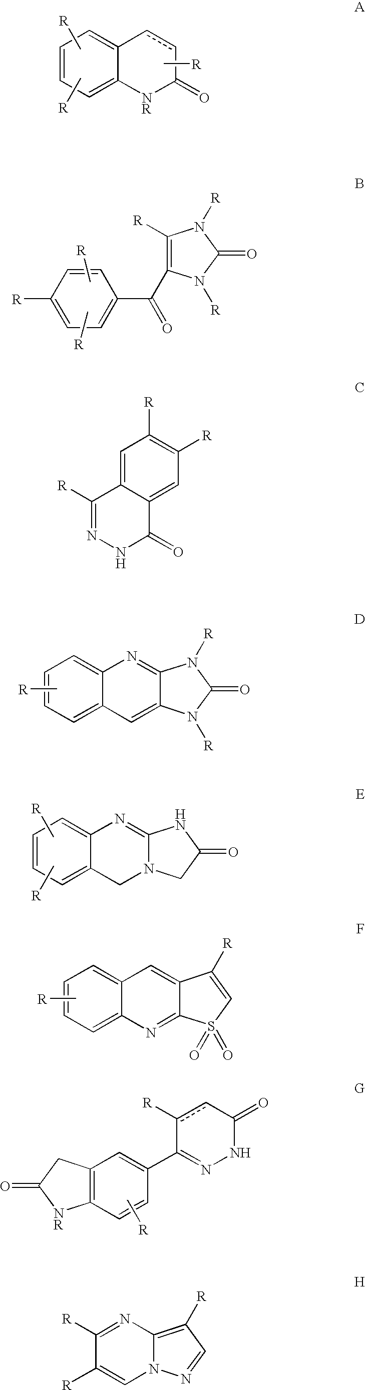 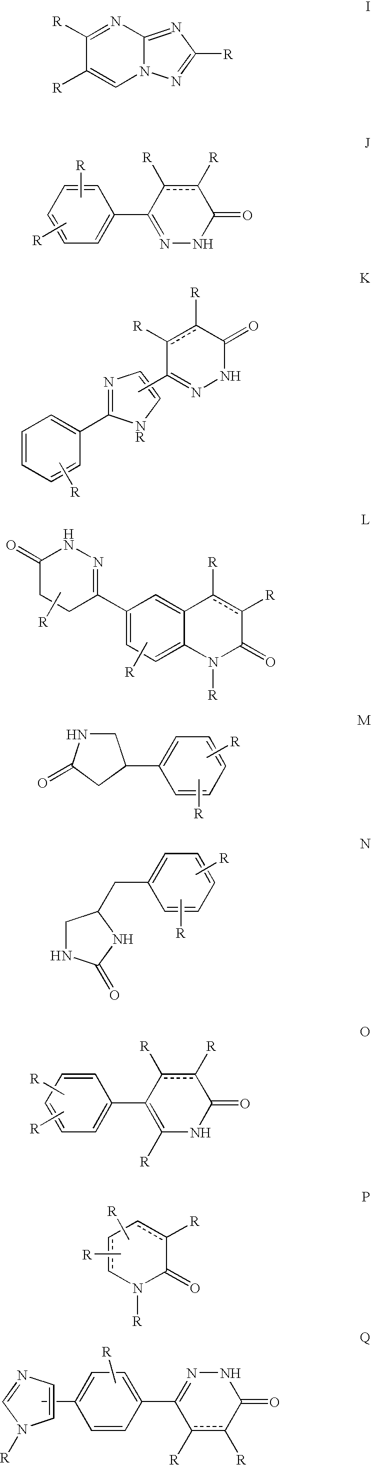
with X bonded to L through any one R group; and

each R group is independently a direct bond, hydrogen, halo, nitro, cyano, trifluoromethyl, amino, NR⁵R⁶, C₁-C₄ alkoxy, C₁-C₄ alkylthio, COOR⁷, C₁-C₁₂ alkyl, C₂-C₁₂ alkenyl or C₂-C₁₂ alkynyl, wherein one or more carbon(s) of said alkyl, alkenyl or alkynyl is optionally replaced with O, S, SO₂ or NR⁵, and/or optionally substituted with carbonyl oxygen or hydroxyl;

with the following provisos:

(a) when Ar is unsubstituted or substituted phenyl, n is 1, R¹ is hydrogen and X is moiety O, then moiety O is not 5-phenyl-6-methyl-2-oxo-1,2-dihydro-3-pyridinecarbonitrile;

(b) when Ar is unsubstituted or substituted phenyl, n is 1 and R¹ is hydrogen or methyl, then X is not moiety J; and

(c) when Ar is substituted phenyl, n is 0 and R¹ is C₁-C₄ alkyl, then X is not moiety A.

Every variable substituent is defined independently at each occurrence. Thus, the definition of a variable substituent in one part of a formula is independent of its definition(s) elsewhere in that formula and of its definition(s) in other formulas.

In one embodiment, Ar is phenyl, naphthyl, pyridyl, isoxazolyl, pyridyl, quinolyl, isoquinolyl, Ar¹, Ar², Ar³, Ar⁴, Ar⁵, Ar⁶ or Ar⁷ 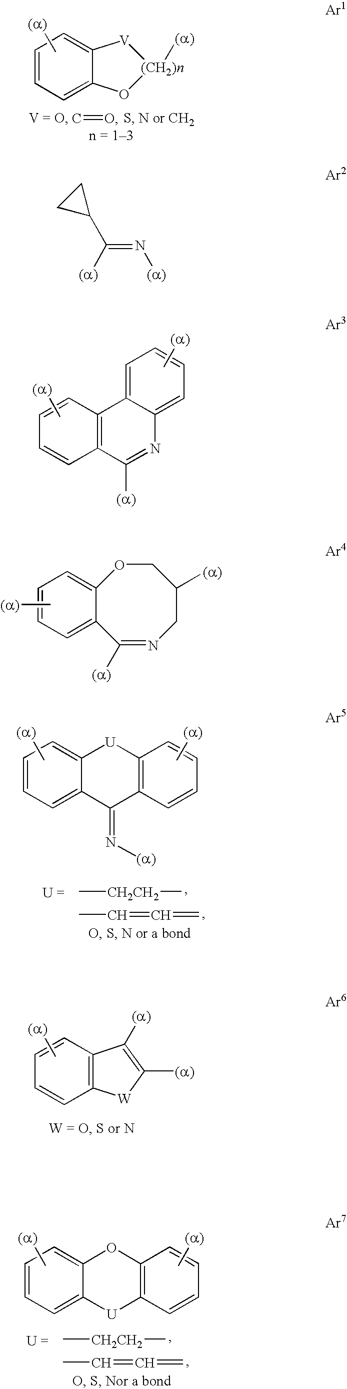
wherein (α) indicates the position where Ar may bond.

In another embodiment, Ar is substituted phenyl, n is 1, R¹ is hydrogen and X is moiety A, B or E.

Since the compounds of the present invention may possess one or more asymmetric carbon center(s), they may be capable of existing in the form of optical isomers as well as in the form of racemic or non-racemic mixtures of optical isomers. The optical isomers can be obtained by resolution of the racemic mixtures according to conventional processes. One such process entails formation of diastereoisomeric salts by treatment with an optically active acid or base, then separation of the mixture of diastereoisomers by crystallization, followed by liberation of the optically active bases from the salts. Examples of appropriate acids are tartaric, diacetyltartaric, dibenzoyltartaric, ditoluoyltartaric and camphorsulfonic acid.

A different process for separating optical isomers involves the use of a chiral chromatography column optimally chosen to maximize the separation of the enantiomers. Still another available process involves synthesis of covalent diastereoisomeric molecules, for example, esters, amides, acetals and ketals, by reacting the inventive compounds with an optically active acid in an activated form, an optically active diol or an optically active isocyanate. The synthesized diastereoisomers can be separated by conventional means such as chromatography, distillation, crystallization or sublimation, and then hydrolyzed to deliver the enantiomerically pure compound. In some cases hydrolysis to the “parent” optically active drug is not necessary prior to dosing the patient, since the compound can behave as a prodrug. The optically active compounds of the present invention likewise can be obtained by utilizing optically active starting materials.

It is understood that the compounds of the present invention encompass individual optical isomers as well as racemic and non-racemic mixtures. In some non-racemic mixtures, the R configuration may be enriched while in other non-racemic mixtures, the S configuration may be enriched.

Examples of a compound of formula I include without limitation:

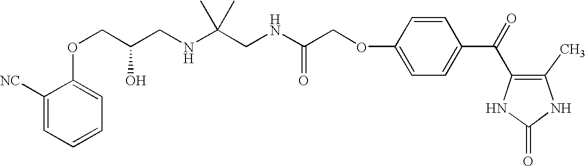

### N-(2-{[(2S)-3-(2-cyanophenoxy)-2-hydroxypropyl]amino}-2-methylpropyl)-2-{4-[(5-methyl-2-oxo(4-imidazolin-4-yl))carbonyl]phenoxy}acetamide

### EXAMPLE 1

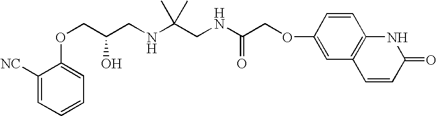

### N-{2-[3-(2-cyano-phenoxy)-2(S)-hydroxy-propylamino]-2-methyl-propyl}-2-(2-oxo-1,2-dihydro-quinolin-6-yloxy)-acetamide

### EXAMPLE 2

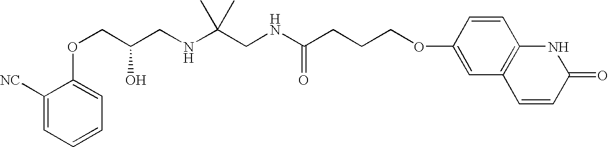

### N-{2-[3-(2-cyano-phenoxy)-2-hydroxy-propylamino]-2-methyl-propyl}-4-(2-oxo-1,2-dihydro-quinolin-6-yloxy)-butyramide

### EXAMPLE 3

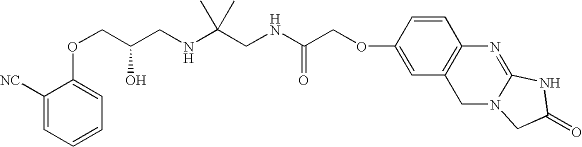

### N-(2-{[(2S)-3-(2-cyanophenoxy)-2-hydroxypropyl]amino}-2-methylpropyl)-2-(2-oxo(4,3a-dihydroimidazolidino[2,1-b]quinazolin-6-yloxy))acetamide

### EXAMPLE 4

### Methods of Use

The present invention further provides a method for regulating calcium homeostasis, comprising administering an effective amount of a compound of the present invention to an animal in need of such regulation.

The present invention further provides a method for treating a disease, disorder or condition in which disregulation of calcium homeostasis is implicated, comprising administering an effective amount of a compound of the present invention to an animal in need of such treatment.

The present invention further provides a method for treating a cardiovascular disease, stroke, epilepsy, an ophthalmic disorder or migraine, comprising administering an effective amount of a compound of the present invention to an animal in need of such treatment.

In one embodiment of the inventive method, the cardiovascular disease is heart failure, hypertension, SA/AV node disturbance, arrythmia, hypertrophic subaortic stenosis or angina. In another embodiment of the inventive method, the heart failure is chronic heart failure or congestive heart failure.

The present invention further provides a method for inhibiting a β-adrenergic receptor and/or PDE, including PDE-3, comprising administering an effective amount of a compound of the present invention to an animal in need of such treatment.

The compound of the present invention may be administered by any means known to an ordinarily skilled artisan. For example, the compound of the present invention may be administered orally, parenterally, by inhalation spray, topically, rectally, nasally, buccally, vaginally, or via an implanted reservoir. The term “parenteral” as used herein includes subcutaneous, intravenous, intramuscular, intraperitoneal, intrathecal, intraventricular, intrasternal, intracranial, and intraosseous injection and infusion techniques. The exact administration protocol will vary depending upon various factors including the age, body weight, general health, sex and diet of the patient; the determination of specific administration procedures would be routine to an ordinarily skilled artisan.

The compound of the present invention may be administered by a single dose, multiple discrete doses or continuous infusion. Pump means, particularly subcutaneous pump means, are useful for continuous infusion.

Dose levels on the order of about 0.001 mg/kg/d to about 10,000 mg/kg/d of the compound of the present invention are useful for the inventive methods, with preferred levels being about 0.1 mg/kg/d to about 1,000 mg/kg/d, and more preferred levels being about 1 mg/kg/d to about 100 mg/kg/d. The specific dose level for any particular patient will vary depending upon various factors, including the activity and the possible toxicity of the specific compound employed; the age, body weight, general health, sex and diet of the patient; the time of administration; the rate of excretion; the drug combination; the severity of the congestive heart failure; and the form of administration. Typically, in vitro dosage-effect results provide useful guidance on the proper doses for patient administration. Studies in animal models are also helpful. The considerations for determining the proper dose levels are well known in the art and within the skill of a physician.

Any administration regimen well known to an ordinarily skilled artisan for regulating the timing and sequence of drug delivery can be used and repeated as necessary to effect treatment in the inventive method. The regimen may include pretreatment and/or co-administration with additional therapeutic agents.

The compound of the present invention can be administered alone or in combination with one or more additional therapeutic agent(s) for simultaneous, separate, or sequential use. The additional agent(s) may be any therapeutic agent(s), including without limitation one or more compound(s) of the present invention. The compound of the present invention can be co-administered with one or more therapeutic agent(s) either (i) together in a single formulation, or (ii) separately in individual formulations designed for optimal release rates of their respective active agent.

### Pharmaceutical Compositions

The present invention further provides a pharmaceutical composition comprising:

(i) an effective amount of a compound of the present invention; and

(ii) a pharmaceutically acceptable carrier.

The inventive pharmaceutical composition may comprise one or more additional pharmaceutically acceptable ingredient(s), including without limitation one or more wetting agent(s), buffering agent(s), suspending agent(s), lubricating agent(s), emulsifier(s), disintegrant(s), absorbent(s), preservative(s), surfactant(s), colorant(s), flavorant(s), sweetener(s) and additional therapeutic agent(s).

The inventive pharmaceutical composition may be formulated for administration in solid or liquid form, including those adapted for the following: (1) oral administration, for example, drenches (for example, aqueous or non-aqueous solutions or suspensions), tablets (for example, those targeted for buccal, sublingual and systemic absorption), boluses, powders, granules, pastes for application to the tongue, hard gelatin capsules, soft gelatin capsules, mouth sprays, emulsions and microemulsions; (2) parenteral administration, for example, by subcutaneous, intramuscular, intravenous or epidural injection as, for example, a sterile solution or suspension, or a sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

### EXAMPLES

Synthesis of Compounds

As shown in general Scheme I below, protected aryl hydroxyl precursors of moiety X (1) (P¹ may be, for example, acetyl, benzyl, alkylsilyl or another appropriate protecting group and Q¹, R, S and T are selected to reach a particular moiety X) may be deprotected and converted to carboxyalkoxy acids (3), which may then be reacted with substituted N-(aminoalkyl)-2-hydroxy-3-phenoxypropylamines (5) using standard coupling conditions for formation of amide bonds. The substituted N-(aminoalkyl)-2-hydroxy-3-phenoxypropylamines may in turn be synthesized by reacting substituted 3-phenoxy-1,2-epoxypropanes (4) with monoprotected alkyldiamines followed by removal of the nitrogen protecting group. Epoxides (4) are commercially available or may be readily prepared by standard synthetic methods well known to those skilled in the art of organic synthesis, such as by reacting a phenol with epichlorohydrin. Enantiomerically pure epoxides (4) may be prepared, for example, by the methodology described in Sharpless et al., J. Org. Chem. 1989, 54, 1295-1304. The use and removal of amine and oxygen protecting groups are described in detail in PROTECTIVE GROUPS IN ORGANIC SYNTHESIS by T. W. Greene, J. Wiley and Sons.

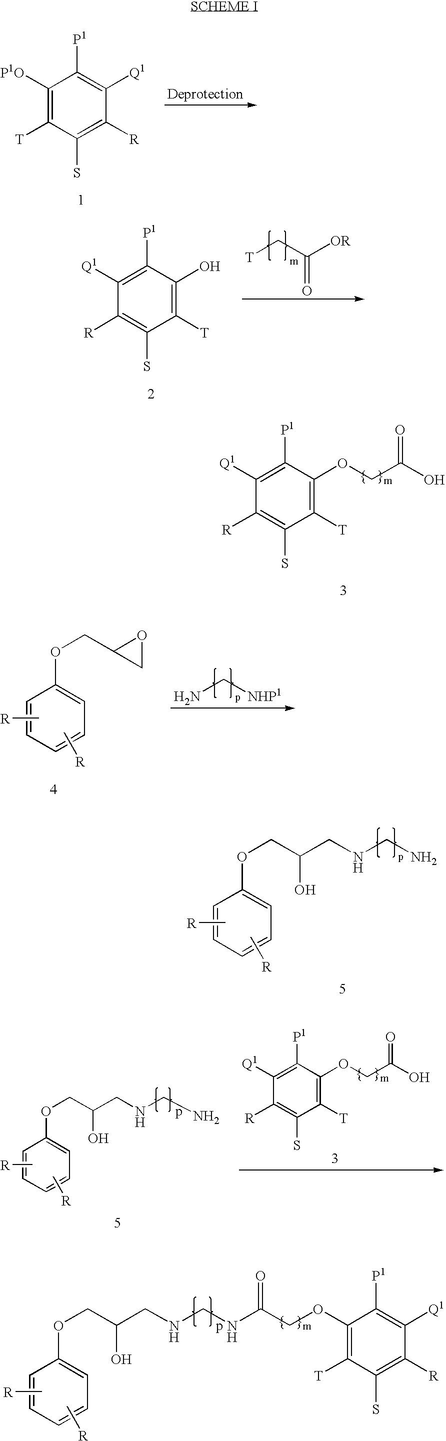

Alternatively, carboxyalkoxy compounds (3) can be reacted with the protected alkyldiamines and subsequently deprotected to produce intermediate compounds (6) in Scheme II below. Reaction of intermediate compounds (6) with epoxides (4) under standard conditions yields the final products.

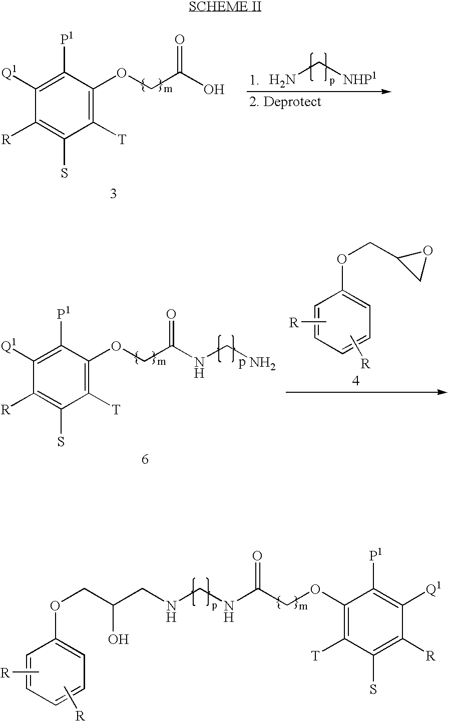

### Example 1

N-(2-{[(2S)-3-(2-cyanophenoxy)-2-hydroxypropyl]amino}-2-methylpropyl)-2-{4-[(5-methyl-2-oxo(4-imidazolin-4-yl))carbonyl]phenoxy}acetamide (Example 1) is synthesized according to Scheme III.

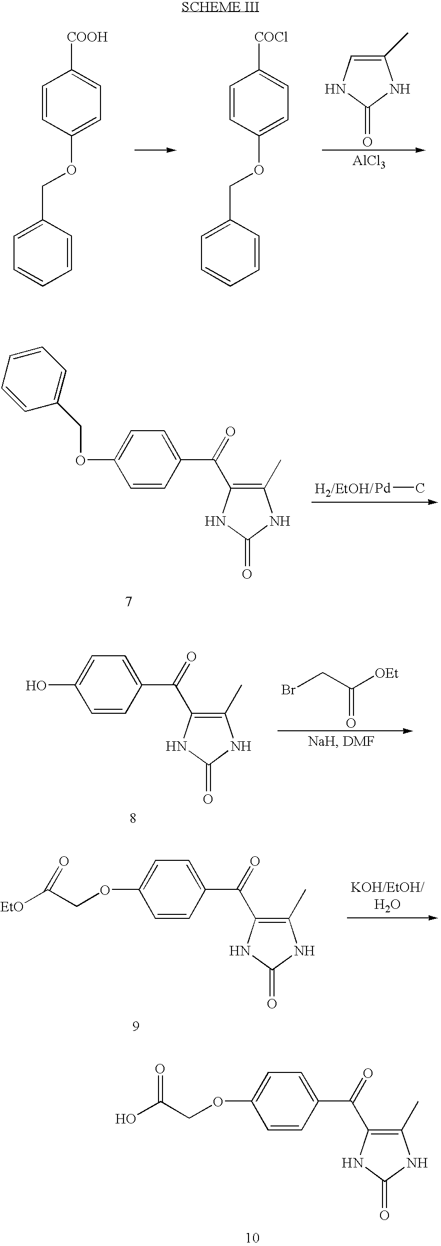

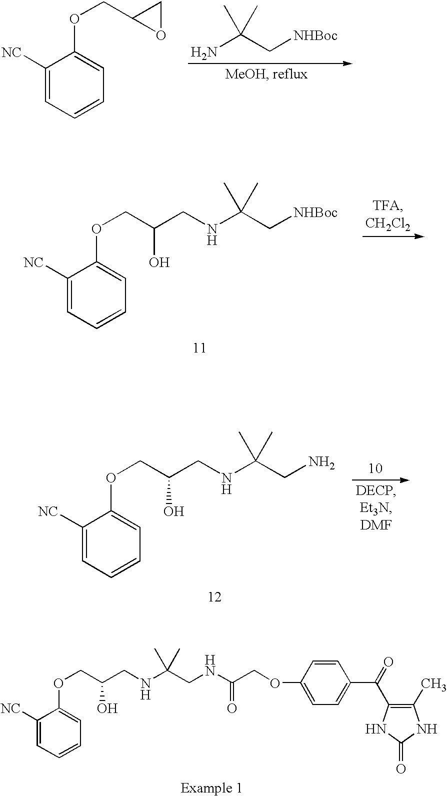

4-Methyl-5-{[4-(phenylmethoxy)phenyl[carbonyl}-4-imidazolin-2-one (7): The potassium salt of 4-(phenylmethoxy)benzoic acid (56 mmol) is suspended in 150 mL of CH₂Cl₂, cooled in an ice-bath, and treated with 7.50 g (60 mmol) of oxalyl chloride added dropwise. Following the completion of the addition, the mixture is refluxed for 30 minutes, cooled and filtered. The filtrate is added dropwise to a stirred mixture of 4-methyl-4-imidazolin-2-one (56 mmol, prepared by the method described in Duschinsky et al., J. Am. Chem. Soc. 1945, 67, 2079) and anhydrous aluminum chloride (112 mmol) in 50 mL of nitrobenzene. The resulting mixture is stirred at 65° C. for 6 hours and then poured over ice. The precipitate formed is collected by filtration, washed with ether and water, and recrystallized from ethanol/water to yield the benzyl protected compound (7).

5-[(4-Hydroxyphenyl)carbonyl]-4-methyl-4-imidazolin-2-one (8): Compound 7 (15 mmol) is dissolved in ethanol, treated with a catalytic amount of 10% palladium on carbon, and hydrogenated at 50 psi overnight. The catalyst is removed by filtration and the solvent is removed in vacuo to yield compound 8 as an oil.

Ethyl 2-{4-[(5-methyl-2-oxo-4-imidazolin-4-yl)carbonyl]phenoxy}acetate (9): Compound 8 (10 mmol) is added portionwise to a stirred mixture of 60% NaH in mineral oil (20 mmol NaH) in 60 mL of dry dimethylformamide (DMF), under an inert atmosphere. The reaction is stirred for 30 minutes after hydrogen evolution ceases, cooled to 0° C., and treated with ethyl bromoacetate (12 mmol) in DMF (5 mL). The reaction mixture is stirred at 0° C. for 30 minutes, allowed to come to room temperature, and finally heated to 85° C. for an hour. After cooling, the volatiles are removed in vacuo and the residue is dissolved in 200 mL of ethyl acetate. The organic phase is washed with water, dried, concentrated under reduced pressure, and the crude product is purified on a silica gel column, eluting with 2% methanol in methylene chloride, to deliver compound 9.

2-{4-[(5-Methyl-2-oxo-4-imidazolin-4-yl)carbonyl]phenoxy}acetic acid (10): A solution of compound 9 (3 mmol) in 30 mL of 1:1 ethanol:water containing potassium hydroxide (9 mmol) is heated to 80° C., with stirring and under an inert atmosphere, for 2 hours. After cooling, the reaction mixture is diluted with 1 00 mL of water and extracted with 2×150 mL of ether. The aqueous phase is cooled in an ice bath and treated with 6N HCl to pH 2. The precipitate formed is collected via vacuum filtration, washed with water, and dried under vacuum at 80° C. to deliver compound 10 as a white solid.

(tert-Butoxy)-N-(2-{[3-(2-cyanophenoxy)-2-hydroxypropyl]amino}-2-methylpropyl)carboxamide (11): A mixture of 2-(oxiran-2-ylmethoxy)benzenecarbonitrile (10 mmol) and N-(2-amino-2-methylpropyl)(tert-butoxy)carboxamide (10 mmol) in methanol (25 mL) is refluxed under inert atmosphere for 5 hours, cooled and concentrated in vacuo. Purification of the residue on a silica gel column, eluting with 2% methanol in methylene chloride, delivers the Boc-protected intermediate 11 as a white solid.

2-{3-[(2-Amino-tert-butyl)amino]-2-hydroxypropoxy}benzenecarbonitrile (12): The protecting group is removed by dissolving compound 11 (5 mmol) in methylene chloride (15 mL), cooling to 0° C., and treating with 15 mL of trifluoroacetic acid. The mixture is stirred for 1 hour at 0° C., 2 hours at room temperature, and then concentrated under vacuum. The residue is dissolved in 50 mL of acetonitrile, treated with anhydrous potassium carbonate (20 mmol). After stirring at 60° C. for 2 hours, the mixture is filtered and the filtrate is concentrated and purified on a silica gel column, eluting with 2% methanol in methylene chloride to produce the free amine 12 as an oil.

N-(2-{[(2S)-3-(2-cyanophenoxy)-2-hydroxpropyl]amino}-2-methylpropyl)-2-{4-[(5-methyl-2-oxo(4-imidazolin-4-yl))carbonyl]phenoxy}acetamide (Example 1): A mixture of compound 12 (2.5 mmol), compound 11 (2.3 mmol) and diethylcyanophosphonate (2.5 mmol) in 15 mL of DMF is cooled to 0° C. and treated with triethylamine (5 mmol) in 3 mL of DMF. After being allowed to come to room temperature, the mixture is stirred overnight. After concentration under reduced pressure, the residue is purified on a silica gel column, eluting with 2% methanol in methylene chloride to yield the compound of Example 1 as a solid.

### Example 2

N-{2-[3-(2-cyano-phenoxy)-2(S)-hydroxy-propylamino]-2-methyl-propyl}-2-(2-oxo-1,2-dihydro-quinolin-6-yloxy)-acetamide (Example 2) is synthesized according to Scheme IV.

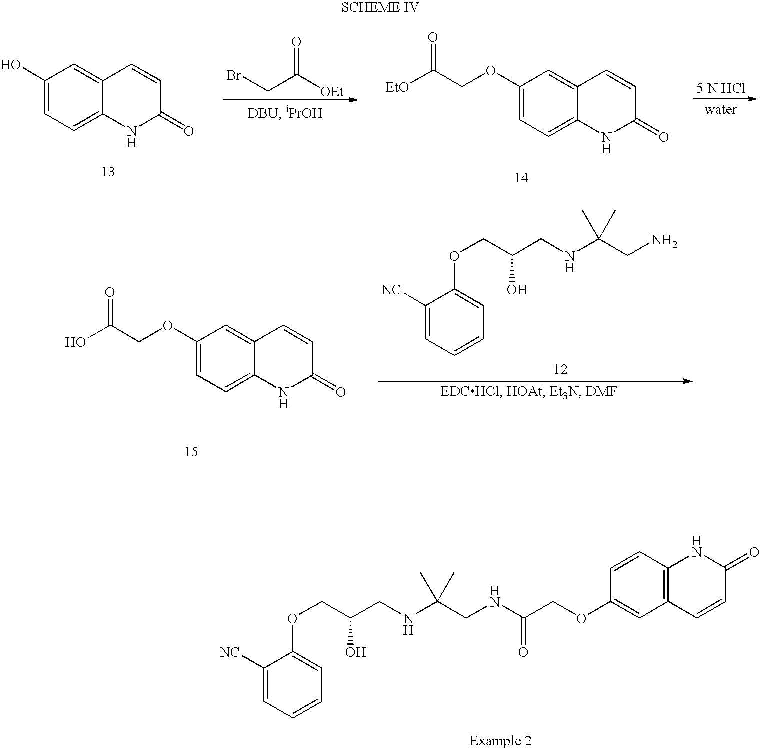

(2-Oxo-1,2-dihydro-quinolin-6-yloxy)-acetic acid (15) via (2-Oxo-1,2-dihydro-quinolin-6-yloxy)-acetic acid ethyl ester (14): To a stirred solution of 6-hydroxy-1H-quinolin-2-one (13, 500 mg, 3.10 mmol) and 1,8-diazabicycle[5.4.0]undec-7-ene (935 μL, 6.25 mmol) in 2-propanol (50 mL) was added dropwise ethyl bromoacetate (688 μL, 6.20 mmol) at ambient temperature. The mixture was stirred for 18 hours at 85° C., allowed to cool to ambient temperature and evaporated to dryness. The dark oily residue (crude 4-(2-oxo-1,2-dihydro-quinolin-6-yloxy)-acetic acid ethyl ester, 14) was suspended in aqueous hydrochloric acid (5 N, 25 mL), stirred for 2 hours at 85° C. and allowed to cool to ambient temperature. The formed precipitate was filtered off with suction, rinsed with water (50 mL), n-hexane (50 mL) and diethylether (50 mL), and dried under vacuum to give 4-(2-oxo-1,2-dihydro-quinolin-6-yloxy)-acetic acid (15) as light brown powder (204 mg, 30% yield, >90% pure by LC-MS and ¹H NMR).

N-{2-[3-(2-Cyano-phenoxy)-2(S)-hydroxy-propylamino]-2-methyl-propyl}-2-(2-oxo-1,2-dihydro-quinolin-6-yloxy)-acetamide (Example 2): To a stirred solution of (2-oxo-1,2-dihydro-quinolin-6-yloxy)-acetic acid (15, 78 mg, 0.357 mmol), 1-(3-dimethylaminopropyl)-3-ethyl carbodiimide hydrochloride (EDC.HCl, 68 mg, 0.357 mmol) and 7-hydroxyazabenzotriazole (HOAt, 48.5 mg, 0.357 mmol) in N,N-dimethylformamide (1.5 mL) under N₂ were added a solution of 2-[3-(2-amino-1,1-dimethyl-ethylamino)-2(S)-hydroxy-propoxy]-benzonitrile (12, 120 mg, 0.357 mmol) in N,N-dimethylformamide (1.5 mL) and triethylamine (110 μL, 0.788 mmol). The reaction mixture was stirred at ambient temperature for 3 hours, then poured into saturated brine (10 mL), made strongly alkaline (pH about 11-12) with aqueous 2N sodium hydroxide solution and extracted with ethyl acetate (3×20 mL). The combined organic layers were washed with H₂O (20 mL), dried (MgSO₄) and concentrated under reduced pressure. The residue was purified by flash column chromatography over silica gel (3 g) eluting with dichloromethane/methanol (9:1). Fractions with R_{f}=0.14 were combined and concentrated under reduced pressure to give N-{2-[3-(2-cyano-phenoxy)-2(S)-hydroxy-propylamino]-2-methyl-propyl}-2-(2-oxo-1,2-dihydro-quinolin-6-yloxy)-acetamide (Example 2) as an off-white solid (56 mg, 34% yield, 97% pure by LC-MS and ¹H-NMR).

### Example 3

N-{2-[3-(2-cyano-phenoxy)-2-hydroxy-propylamino]-2-methyl-propyl}-4-(2-oxo-1,2-dihydro-quinolin-6-yloxy)-butyramide was synthesized according to Scheme IV by replacing ethyl bromoacetate in the first step with ethyl 4-bromobutyrate. Example 3 was obtained as an off-white solid (53 mg, 97% pure by LC-MS and ¹H-NMR).

### Example 4

N-(2-{[(2S)-3-(2-cyanophenoxy)-2-hydroxypropyl]amino}-2-methylpropyl)-2-(2-oxo(4,3a-dihydroimidazolidino[2,1-b]quinazolin-6-yloxy))acetamide is synthesized according to Scheme V.

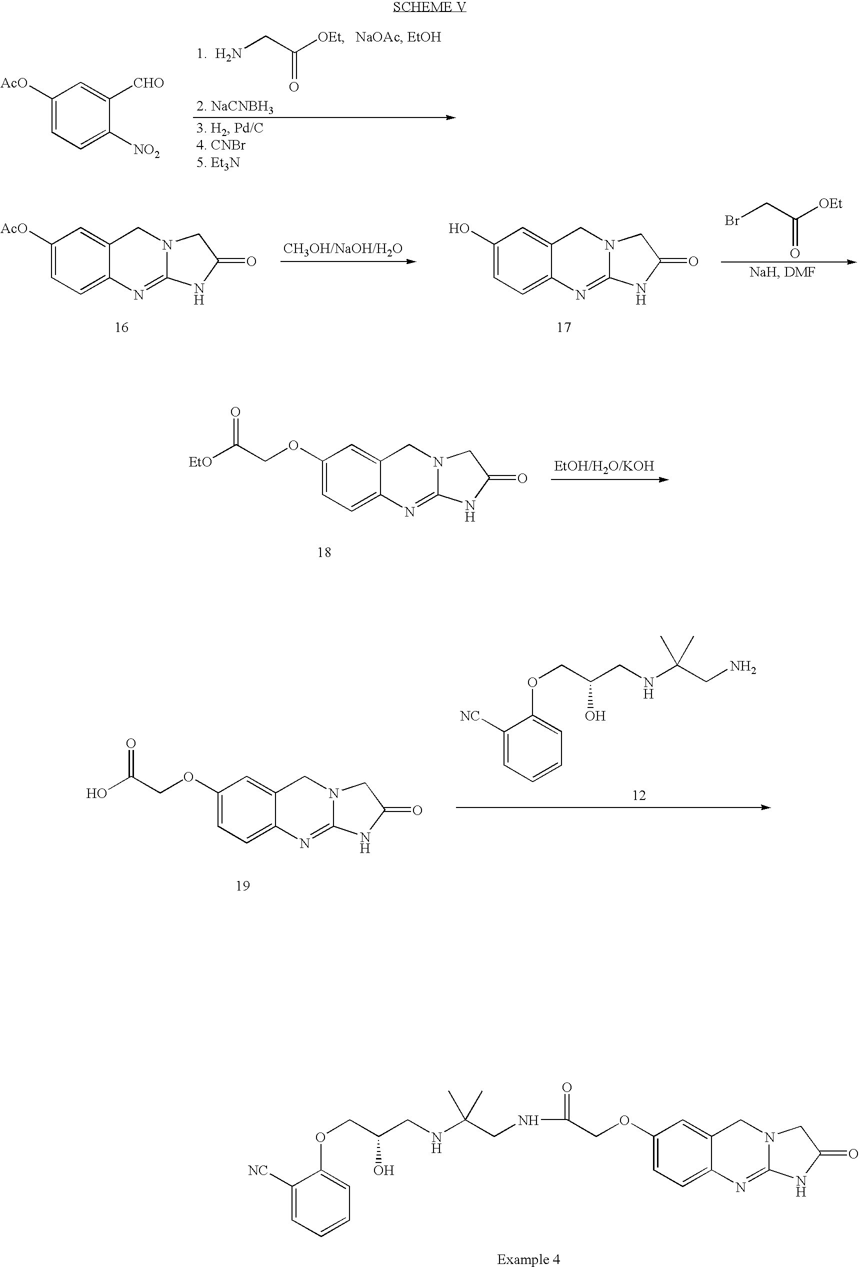

2-Oxo-4,3a-dihydroimidazolidino[2,1-b]quinazolin-6-yl acetate (16): 3-Formyl-4-nitrophenyl acetate (10 mmol) is added to a solution prepared from glycine ethyl ester hydrochloride (3.0 g, 24 mmol) and anhydrous sodium acetate (820 mg, 10 mmol) in methanol (80 mL). After stirring the thick mixture for 15 minutes, sodium cyanoborohydride (380 mg, 6 mmol) is added, resulting in dissolution of the precipitate. After stirring for an hour, the solvent is evaporated and the residue is partitioned between ethyl acetate (50 mL) and saturated aqueous NaHCO₃ (50 mL). The layers are separated and the aqueous phase is extracted with additional ethyl acetate. The combined organic fractions are washed with saturated aqueous NaHCO₃ and brine, dried over magnesium sulfate, and concentrated in vacuo. The crude residue is purified by silica gel chromatography to furnish the benzylamine intermediate, which is dissolved in 20 mL of ethanol and hydrogenated at 60 psi over 10% Pd—C overnight. After removing the catalyst by filtration, a solution of cyanogen bromide (760 mg; 7.1 mmol) in 5 mL of ethanol is added to the filtrate. After stirring overnight, the mixture is treated with triethylamine (1.1 mL, 7.8 mmol) and stirring is continued overnight again. The formed precipitate is collected by filtration, washed repeatedly with water and ethanol-ether, and dried to provide compound 16.

6-Hydroxy-4,3a-dihydroimidazolidino[2,1-b]quinazolin-2-one (17): Compound 16 (5 mmol) is suspended in 10 mL of methanol and treated with 2 mL of a 2.5 M solution of NaOH. After stirring for 1 hour, the precipitate is collected by filtration, washed with acetone, and dried under vacuum to furnish compound 17 as a solid.

Ethyl 2-(2-oxo-4,3a-dihydroimidazolidino[2,1-b]quinazolin-6-yloxy)acetate (18): Compound 17 (15 mmol) is added portionwise to a stirred mixture of 60% NaH in mineral oil (30 mmol NaH) in 90 mL of dry DMF, under inert atmosphere. The reaction is stirred for 30 minutes after hydrogen evolution ceases, cooled to 0° C., and treated with ethyl bromoacetate (18 mmol) in DMF (8 mL). The reaction mixture is stirred at 0° C. for 30 minutes, allowed to come to room temperature, and finally heated to 85° C. for an hour. After cooling, the volatiles are removed in vacuo and the residue is dissolved in 300 mL of ethyl acetate. The organic phase is washed with water, dried, concentrated under reduced pressure, and the crude product is purified on a silica gel column, eluting with 5% methanol in methylene chloride, to deliver compound 18.

2-(2-Oxo-4,3a-dihydroimidazolidino[2,1-b]quinazolin-6-yloxy)acetic acid (19): A solution of compound 18 (7.5 mmol) in 75 mL of 1:1 ethanol:water containing potassium hydroxide (23 mmol) is heated to 80° C., with stirring and under an inert atmosphere, for 2 hours. After cooling, the reaction mixture is diluted with 230 mL of water and extracted with 3×200 mL of ether. The aqueous phase is cooled in an ice bath and treated with 6 N HCl to pH 2. The precipitate formed is collected via vacuum filtration, washed with water, and dried under vacuum at 80° C. to deliver compound 19 as a white solid.

N-(2-{[3-(2-cyanophenoxy)-2-hydroxypropyl]amino}-2-methylpropyl)-2-(2-oxo(4,3a-dihydroimidazolidino[2,1-b]quinazolin-6-yloxy))acetamide (Example 4): A mixture of compound 12 (5 mmol), compound 19 (4.6 mmol) and diethylcyanophosphonate (5 mmol) in 30 mL of DMF is cooled to 0° C. and treated with triethylamine (10 mmol) in 7 mL of DMF. After being allowed to come to room temperature, the mixture is stirred overnight. After concentration under reduced pressure, the residue is purified on a silica gel column, eluting with 2% methanol in methylene chloride, to furnish the compound of Example 4 as a solid.

PDE-3 Inhibitory Activity

### Example 5

### Assay for Measuring cAMP PDE-3 Inhibitory Activity

Human platelet cyclic AMP phosphodiesterase is prepared according to the method of Alvarez et al., Mol. Pharmacol. 29: 554 (1986). The PDE incubation medium contains 10 mM Tris-HCl (2-amino-2-(hydroxymethyl)-1,3-propanediol, hydrochloride) buffer, pH 7.7, 10 mM MgSO₄, and 1 □M [³H]AMP (0.2 □Ci) in a total volume of 1.0 mL. Test compounds are dissolved in DMSO immediately prior to addition to the incubation medium, and the resulting mixture is allowed to stand for 10 minutes prior to the addition of enzyme. Following the addition of PDE, the contents are mixed and incubated for 10 minutes at 30° C. Three assays are performed for each of five test compound concentrations, the mean of the determinations (n=3) at each concentration is plotted, and IC₅₀ values are determined graphically.

P-Adrenergic Receptor Binding Activity

### Example 6

### Radioligand for Measuring β₁-Receptor Affinity

β₁-Adrenergic receptor binding is measured in human recombinant beta-1 receptors expressed in CHO-REX16 cells, using [¹²⁵I] (−) iodocyanopindolol (2000 Ci/mmol) as the radioligand, as described in Kalaria et al., J. Neurochem. 53: 1772-81 (1998), and Minneman et al., Mol. Pharmacol. 16: 34-46 (1979).

### Example 7

### Radioligand for Measuring β₂-Receptor Affinity

β₂-Adrenergic receptor binding is measured in human recombinant beta-2 receptors expressed in CHO-WT21 cells, using [¹²⁵I] (−) iodocyanopindolol (2000 Ci/mmol) as the radioligand, as described in Kalaria et al. (1998) and Minneman et al. (1979), supra.

Restoration of Calcium Homeostasis in Heart Tissue

### Example 8

### Assay for Measuring Contraction-Relaxation in Guinea Pig Papillary Muscle

Male guinea pigs (400-500 g) are killed by cervical dislocation and the hearts are quickly removed, immersed in ice-cold, and oxygenated in Kreb's solution containing 113.1 mM NaCl, 4.6 mM KCl, 2.45 mM CaCl₂, 1.2 mM MgCl₂, 22.0 mM NaH₂PO₄, and 10.0 mM glucose; pH 7.4 with 95% O₂-5% CO₂. The ventricles are opened and papillary muscles are removed with the chordae tandineae and a base of surrounding tissue intact. The tendinous ends of the muscles are ligated with silk thread, and the muscles are mounted in vertical, double-jacketed organ baths containing 10 mL of oxygenated Kreb's solution kept at 37° C. The tendinous end is attached to a Grass isometric force transducer, while a metal hook is inserted into the base of the muscle.

Following a 45 minute equilibration period under a 1 gram tension, control contractions are elicited by stimulating the muscle using stainless steel field electrodes at a frequency of 1.0 Hz, 2.0 ms duration. The amplitude of the stimulus is adjusted to be approximately 1.5 times the threshold amplitude sufficient to elicit a contraction of the tissues. Control contraction-relaxation cycles are recorded for 30 seconds continuously. Cumulative test drug concentrations are then injected directly into the bath while the tissue is being stimulated. Contraction-relaxation recordings are made continuously, for 30 seconds per test compound concentration. A series of washout contractions is recorded following a change of solution. Provided that the amplitude of contraction returns to that measured in control conditions, a single concentration of positive control is then tested on the tissue in the same manner as the test compound.

Contraction amplitude as well as the time courses of contraction and relaxation are quantified. All recordings are normalized against control values; statistical analysis of the results is made using t-tests or ANOVAs.

All publications, patents and patent applications identified above are herein incorporated by reference.

The invention being thus described, it will be apparent to those skilled in the art that the same may be varied in many ways without departing from the spirit and scope of the invention. Such variations are included within the scope of the invention to be claimed.

1. A compound of formula I: 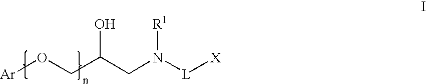
or a pharmaceutically acceptable equivalent, an isomer or a mixture of isomers thereof, wherein:
n is 0 or 1;
Ar is an aryl or heteroaryl radical, which aryl or heteroaryl radical is optionally substituted with 1 to 3 substituent(s) selected from R², R³ and R⁴;
R¹ is hydrogen, C₁-C₈ alkyl, C₂-C₈ alkenyl, C₂-C₈ alkynyl, C₃-C₈ cycloalkyl or C₃-C₈ cycloalkenyl;
R², R³ and R⁴ are independently cyano, nitro, halogen, hydrogen, trifluoromethyl, acylaminoalkyl, NR⁵, —NHSO₂R¹, —NHCONHR¹, C₁-C₄ alkoxy, C₁-C₄ alkylthio, C₁-C₈ alkyl, C₂-C₈ alkenyl or C₂-C₈ alkynyl, wherein one or more carbon(s) of said alkyl, alkenyl or alkynyl chain is/are optionally replaced with O, S, SO₂, or NR⁵, and/or optionally substituted with carbonyl oxygen or hydroxyl;
L is C₁-C₁₂ alkyl, C₂-C₁₂ alkenyl or C₂-C₁₂ alkynyl, wherein one or more carbon(s) of said alkyl, alkenyl or alkynyl is/are optionally replaced with O, S, SO₂, or NR₅, and/or optionally substituted with carbonyl oxygen or hydroxyl;
R⁵ is hydrogen, a lone pair of electrons, C₁-C₈ alkyl, C₂-C₈ alkenyl or C₃-C₈ alkynyl, which alkyl, alkenyl or alkynyl is optionally substituted with phenyl or substituted phenyl;
X is a moiety of formula A, B, C, D, E, F, G, H, I, J, K, L, M, N, O, P or Q 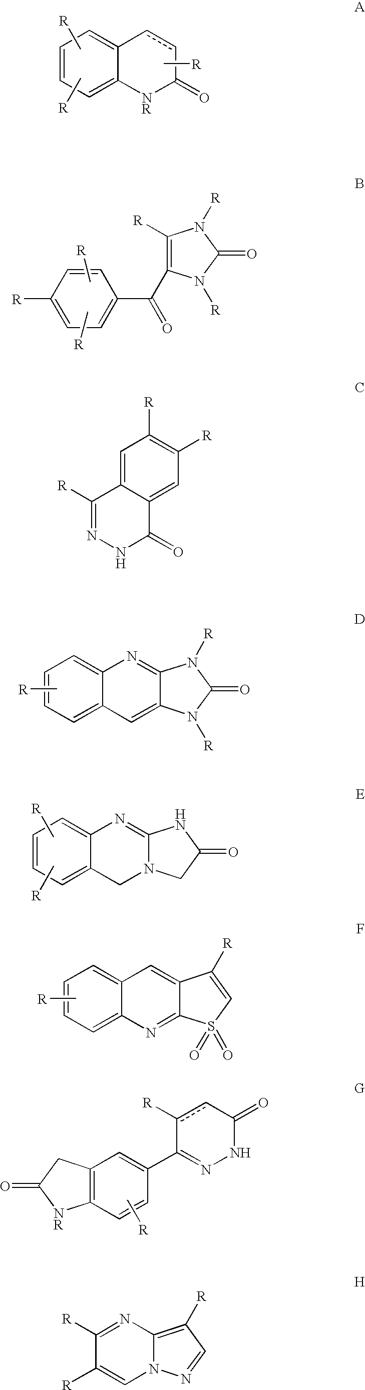 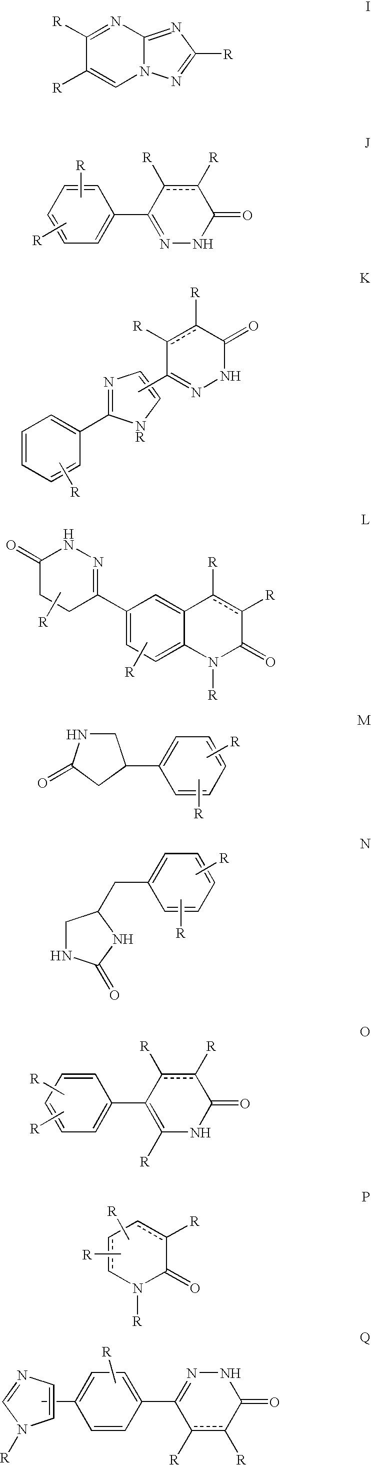
with X bonded to L through any one R group; and
each R group is independently a direct bond, hydrogen, halo, nitro, cyano, trifluoromethyl, amino, NR⁵R⁶, C₁-C₄ alkoxy, C₁-C₄ alkylthio, COOR⁷, C₁-C₁₂ alkyl, C₂-C₁₂ alkenyl or C₂-C₁₂ alkynyl, wherein one or more carbon(s) of said alkyl, alkenyl or alkynyl is optionally replaced with O, S, SO₂ or NR⁵, and/or optionally substituted with carbonyl oxygen or hydroxyl;
with the following provisos:
(a) when Ar is unsubstituted or substituted phenyl, n is 1, R¹ is hydrogen and X is moiety O, then moiety O is not 5-phenyl-6-methyl-2-oxo-1,2-dihydro-3-pyridinecarbonitrile;
(b) when Ar is unsubstituted or substituted phenyl, n is 1 and R¹ is hydrogen or methyl, then X is not moiety J; and
(c) when Ar is substituted phenyl, n is 0 and R¹ is C₁-C₄ alkyl, then X is not moiety A.

2.-32. (canceled)
